(19) United States
(12) Patent Application Publication
(10) Pub. No.: US 2023/0330025 A1
(43) Pub. Date: Oct. 19, 2023

(54) Particle Modifiers for Multi-Drug Loaded Nanoparticles

(71) Applicant: UNIVERSITY OF CONNECTICUT, Farmington, CT (US)

(72) Inventors: Gowtham Yenduri, Farmington, CT (US); Diane J. Burgess, Farmington, CT (US); Antonio Costa, Farmington, CT (US)

(21) Appl. No.: 18/301,661

(22) Filed: Apr. 17, 2023

Related U.S. Application Data
(60) Provisional application No. 63/331,816, filed on Apr. 16, 2022.

(51) Int. Cl. A61K 9/16; A61K 9/127

(52) U.S. Cl. CPC A61K 9/1617; A61K 9/127

(57) ABSTRACT

The present disclosure provides therapeutic nanoparticles that include a first layer surrounding a core region, one or more particle modifiers present in or on the first layer, and one or more therapeutics present in the core region. Compositions and methods related to the nanoparticles are also provided.

### CROSS REFERENCE TO RELATED APPLICATIONS

This application claims the benefit of U.S. Provisional Patent Application No. 63/331,816, filed Apr. 16, 2022, which is incorporated by reference herein in its entirety.

### STATEMENT REGARDING FEDERALLY SPONSORED RESEARCH

This invention was made with government support under Grant No. 1U01FD006975 awarded by the Food and Drug Administration. The government has certain rights in the invention.

### BACKGROUND OF THE DISCLOSURE

Chemotherapy is the most commonly used treatment method for a variety of cancers, but it can lead to unnecessary toxicity and drug resistance in nearly half of tumors. Drug resistance contributes to the failure of therapeutics in many different cancers, such as cervical cancer and leukemia, due to mechanisms such as decreased drug uptake and decreased drug-induced cell death. Combination therapy has been shown often to be effective against drug resistance and to improve clinical outcomes for various cancers. However, these therapies are often limited in their effectiveness due to uncoordinated uptake and difficulty entering tumor tissue at the optimal dose leading to a decrease in their synergistic effect. Moreover, synthesis of delivery systems that can theoretically deliver more than one therapeutic is typically laborious and of low yield.

### SUMMARY OF THE DISCLOSURE

The present disclosure provides therapeutic nanoparticles, as well as related methods and compositions. In one aspect, a nanoparticle is provided that includes a first layer surrounding a core region, one or more particle modifiers present in or on the first layer, and one or more therapeutics present in the core region.

In some embodiments, the nanoparticles include polymer-based nanoparticles; whereas, in other embodiments, the nanoparticles include lipid-based nanoparticles, or mixtures of polymer-based and lipid-based nanoparticles.

In another aspect, the disclosure provides a composition including a plurality of nanoparticles, and one or more of a pharmaceutically acceptable carrier, a pharmaceutically acceptable excipient, or a pharmaceutically acceptable diluent.

In another aspect, the disclosure provides a method for treating a cancer, including administering to a subject having cancer an amount of nanoparticles effective to treat the cancer.

In another aspect, the disclosure provides method for making lipid-based nanoparticles or liposomes including dissolving lipid components and one or more particle modifiers (including second therapeutic, or docetaxel (DTX) or a salt thereof) in an organic solvent to produce a lipid-particle modifier-organic solvent solution; injecting the lipid-particle modifier-organic solvent solution into an aqueous solution, forming a turbulent jet at the site where the lipid-organic solvent solution and the aqueous solution mix, forming lipid-based nanoparticles or liposomes; concentrating the lipid-based nanoparticles or liposomes; and mixing the concentrated liposomes with a solution of a first therapeutic or a salt thereof, at an elevated temperature, e.g., about 70° C. or higher, or at about 70° C.

In another aspect, the disclosure provides lipid-based nanoparticles or liposomes made according to the method described in the aspect above.

### BRIEF DESCRIPTION OF THE DRAWINGS

FIG. 1 shows Cryo-TEM images of Lip-DTX (liposomal docetaxel) formulation at two different magnifications.

FIG. 2 shows Cryo-TEM images of Lip-DTX/DOX (liposomal docetaxel+doxorubicin) formulation. Black arrows indicate spherical structures among a predominately elongated population.

FIG. 3 shows a graph of sample peaks of the lipid components that were quantified. HSPC contains two peaks representing the two main lipids that make up its composition (DPPC and DSPC). The DPPC peak was used to quantify the HSPC concentration in the samples.

FIG. 4 shows Calibration curves of DOX (circles) and DTX (squares). The error bars were obtained from triplicate samples.

FIG. 5A (Lip-DTX), FIG. 5B (Lip-DOX), and 5C (Lip-DTX/DOX) are graphs that display the change in particle size (bars) and PDI (line) for the duration of the long-term stability study. FIG. 5D shows relatively no change in the encapsulation efficiency of both active pharmaceutical ingredients (APIs) for the duration of the testing period. Error bars were obtained from triplicate samples.

FIG. 6A through FIG. 6C show cumulative release profiles of Lip-DTX (liposomal docetaxel), Lip-DOX (liposomal doxorubicin), Lip-DTX/DOX (Liposomal docetaxel+doxorubicin), and DOX (doxorubicin) solution at three different pH values, where FIG. 6A is pH 7.4, FIG. 6B is pH 6.5 and FIG. 6C is pH 5.5. A bold, underlined “DOX” or “DTX” in the figure key indicates which release profile is being measured in the co-encapsulated formulation. Error bars were obtained from triplicate samples.

FIG. 7 shows a graph of cell viability of HeLa cells treated with blank liposomes, free DTX/DOX, Lip-DTX, Lip-DOX, and Lip-DTX/DOX for 48 hours. Saline was used as the control. Data were presented as the mean±SD of triplicates. *p=0.0106, **p<0.05, ***p<0.001.

FIG. 8 shows Cell viability of K562 cells treated with blank liposomes, free DTX/DOX, Lip-DTX, Lip-DOX, and Lip-DTX/DOX for 48 hours. Data were presented as the mean±SD of triplicates. *p=0.0289, **p<0.05, ***p<0.001.

### DETAILED DESCRIPTION OF THE DISCLOSURE

Herein, nanoparticles are described that can include more than one therapeutic and that are synthesized via a high yield, high throughput process. The nanoparticles are continuously-manufactured formulation containing two therapeutics in the same delivery vehicle. Moreover, both therapeutics were encapsulated at a high efficiency. Thus, nanoparticles as described herein offer significant advantages over other delivery systems that can include more than one therapeutic. The nanoparticles may also be referred to as “nanospheres.”

A number of terms are introduced below:

The terms “treat,” “treatment,” “treating,” or “amelioration” when used in reference to a disease, disorder or medical condition, refer to therapeutic treatment, as well as prophylactic or preventative measures, wherein the object is to prevent, reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers is reduced. Alternatively, treatment is “effective” if the progression of a disease, disorder or medical condition is reduced or halted. That is, “treatment” includes the improvement of symptoms or markers, but also a slowing or cessation of progress or worsening of symptoms that would be expected in the absence of treatment. Also, “treatment” can mean to pursue or obtain beneficial results, or lower the chances of the individual developing the condition even if the treatment is ultimately unsuccessful. Those in need of treatment include those already with the condition, as well as those prone to have the condition or those in whom the condition is to be prevented.

Likewise, the terms “beneficial results” or “desired results” may include, but are not limited to, lessening or alleviating the severity of the disease condition, preventing the disease condition from worsening, curing the disease condition, preventing the disease condition from developing, lowering the chances of a patient developing the disease condition, decreasing morbidity and mortality, and prolonging a patient's life or life expectancy. As non-limiting examples, “beneficial results” or “desired results” may be alleviation of one or more symptom(s), diminishment of extent of the deficit, stabilized (i.e., not worsening) state of a cancer, delay or slowing of a cancer, and amelioration or palliation of symptoms associated with a cancer.

The terms “disease”, “condition” and “disease condition,” as used herein may include, but are not limited to, any form of neoplastic cell proliferative disorders or diseases. Examples of such disorders include but are not limited to cancer and tumor.

The terms “cancer” or “tumor” as used herein refer to an uncontrolled growth of cells which interferes with the normal functioning of the bodily organs and systems, and/or all neoplastic cell growth and proliferation, and all pre-cancerous and cancerous cells and tissues. A subject that has a cancer or a tumor is a subject having objectively measurable cancer cells present in the subject's body. Included in this definition are benign and malignant cancers, as well as dormant tumors or micrometastases. Cancers that migrate from their original location and seed vital organs can eventually lead to the death of the subject through the functional deterioration of the affected organs. The term “invasive” refers to the ability to infiltrate and destroy surrounding tissue, an example being melanoma, which is an invasive form of skin tumor. As used herein, the term “carcinoma” refers to a cancer arising from epithelial cells. A sarcoma is a cancer that arises from transformed cells of mesenchymal origin. Thus, malignant tumors made of cancerous bone, cartilage, fat, muscle, vascular, or hematopoietic tissues are considered sarcomas. This is in contrast to a malignant tumor originating from epithelial cells, which are termed a carcinoma. Sarcomas are given a number of different names based on the type of tissue that they most closely resemble. For example, osteosarcoma resembles bone, chondrosarcoma resembles cartilage, liposarcoma resembles fat, and leiomyosarcoma resembles smooth muscle. Examples of cancer include, but are not limited to, leukemia, sarcoma, Ewing sarcoma, osteosarcoma, nervous system tumor, brain tumor, nerve sheath tumor, breast cancer, colon cancer, carcinoma, lung cancer, hepatocellular cancer, gastric cancer, pancreatic cancer, cervical cancer, ovarian cancer, liver cancer, bladder cancer, cancer of the urinary tract, thyroid cancer, renal cancer, renal cell carcinoma, carcinoma, melanoma, head and neck cancer, brain cancer, and prostate cancer, including but not limited to androgen-dependent prostate cancer and androgen-independent prostate cancer. Examples of brain tumor include, but are not limited to, benign brain tumor, malignant brain tumor, primary brain tumor, secondary brain tumor, metastatic brain tumor, glioma, glioblastoma multiforme (GBM), medulloblastoma, ependymoma, astrocytoma, pilocytic astrocytoma, oligodendroglioma, brainstem glioma, optic nerve glioma, mixed glioma such as oligoastrocytoma, low-grade glioma, high-grade glioma, supratentorial glioma, infratentorial glioma, pontine glioma, meningioma, pituitary adenoma, and nerve sheath tumor. Nervous system tumor or nervous system neoplasm refers to any tumor affecting the nervous system. A nervous system tumor can be a tumor in the central nervous system (CNS), in the peripheral nervous system (PNS), or in both CNS and PNS. Examples of nervous system tumor include but are not limited to brain tumor, nerve sheath tumor, and optic nerve glioma. Leukemia is a type of cancer of the blood or bone marrow characterized by an abnormal increase of immature white blood cells called “blasts”. Examples of leukemia include but are not limited to acute leukemia, chronic leukemia, lymphocytic leukemia, myelogenous leukemia, acute lymphocytic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), acute promyleocytic leukemia, large granular lymphocytic leukemia, and adult T-cell leukemia.

The terms “administer,” “administering,” “delivery” or “delivering” refer to the placement of a nanoparticle, or composition thereof, as disclosed herein into a subject by a method or route that results in at least partial localization of the agents at a desired site. “Route of administration” may refer to any administration pathway known in the art, including but not limited to aerosol, nasal, oral, transmucosal, transdermal, parenteral, enteral, topical or local. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Through the parenteral route, the compositions may be in the form of solutions or suspensions for infusion or for injection, or as lyophilized powders. Through the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release.

The term “effective amount”, “amount effective” or “therapeutically effective amount” refers to an amount sufficient to affect beneficial or desirable biological and/or clinical results. As will be appreciated by those of ordinary skill in this art, the effective amount of microparticles may vary depending on such factors as the desired biological endpoint, the agent to be delivered, the composition of the first layer surrounding a core region, the target tissue, etc. For example, an effective amount of microparticles containing two complementary chemotherapeutics to be delivered can be the amount that results in a reduction in the size of a tumor or a reduction in the growth rate of a tumor.

The terms “therapeutic” or “therapeutic agent” refer to a compound or molecule that, when present in an effective amount, produces a desired therapeutic effect on a subject in need thereof. The present invention contemplates a broad range of therapeutic agents and their use in conjunction with the liposome compositions, as further described herein.

The term “subject” and “patient” are used interchangeably and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like.

A “subject” can be one who has been previously diagnosed with or identified as suffering from or having a condition in need of treatment (e.g., breast cancer) or one or more complications related to the condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition or one or more complications related to the condition or a subject who does not exhibit risk factors. A “subject in need” of treatment for a particular condition can be a subject suspected of having that condition, diagnosed as having that condition, already treated or being treated for that condition, not treated for that condition, or at risk of developing that condition.

The term “mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The terms “statistically significant” or “significantly” refer to statistical evidence that there is a difference. It is defined as the probability of making a decision to reject the null hypothesis when the null hypothesis is actually true. The decision is often made using a p-value.

The term “nanoparticle”, as used herein, refers to any particle having a diameter of less than about 1000 nm. Nanoparticles can comprise, e.g., one or more natural and/or synthetic lipids, polymers, or mixtures thereof. Disclosed nanoparticles may include nanoparticles having a diameter of about 1 nm to about 1000 nm, 10 nm to about 500 nm, or about 20 nm to about 350 nm, or about 30 nm to about 300 nm, or about 35 nm to about 250 nm, or about 40 nm to about 200 nm, or about 45 nm to about 175 nm, or about 50 nm to about 150 nm.

The term “liposome” encompasses any compartment enclosed by at least one lipid bilayer. The term liposome includes unilamellar structures, which comprise a single lipid bilayer, and multilamellar structures, which comprise more than one lipid bilayer. As used herein “liposomes” can refer to unilamellar vesicles (UV) (and, more specifically, small unilamellar vesicles (SUV) and large unilamellar vesicles (LUV)), double bilayer vesicles (DBV), oligolamellar vesicles (OLV) and multilamellar vesicles (MLV). As used herein, a liposome is a type of nanoparticle.

The term “lipid” refers to lipid molecules that can include fats, waxes, steroids, cholesterol, fat-soluble vitamins, monoglycerides, diglycerides, phospholipids, sphingolipids, glycolipids, cationic or anionic lipids, derivatized lipids, and the like, as described in detail below. Lipids can form micelles, monolayers, and bilayer membranes. The lipids can self-assemble into liposomes.

Recitation of ranges of values herein are merely intended to serve as a shorthand method of referring individually to each separate value falling within the range, unless otherwise indicated herein, and each separate value is incorporated into the specification as if it were individually recited. For example, if a concentration range is stated as 1% to 50% (or degrees, mass amounts, and the like), it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The term “about” means plus or minus 10% of the recited measurement.

Disclosed herein are embodiments of nanoparticles that include a first layer surrounding a core region in which the first layer includes one or more particle modifiers, and the core region includes one or more therapeutics or therapeutic agents. A method to make such nanospheres, compositions including such nanospheres, and methods of treatment using such nanospheres.

In one aspect, a nanoparticle is provided that includes a first layer surrounding a core region, one or more particle modifiers present in or on the first layer, and one or more therapeutic present in the core region.

In some embodiments, the nanoparticle is a lipid-based nanoparticle. Such lipid-based nanoparticles can also be referred to as “liposomes.” Liposomes of the present disclosure may include an aqueous core region, or compartment, enclosed by at least one lipid bilayer, or first layer surrounding a core region. When lipids that include a hydrophilic headgroup are dispersed in water, they can spontaneously form bilayer membranes referred to as “lamellae.” Generally, the lamellae are composed of two monolayer sheets of lipid molecules with their non-polar (hydrophobic) surfaces facing each other and their polar (hydrophilic) surfaces facing the aqueous medium. As noted above, liposomes include unilamellar vesicles, which are include a single lipid bilayer and generally have a diameter in the range of about 20 nm to about 1 μm (including both SUV-class and LUV-class liposomes), or about 10 nm to about 500 nm, or about 20 nm to about 350 nm, or about 30 nm to about 300 nm, or about 35 nm to about 250 nm, or about 40 nm to about 200 nm, or about 45 nm to about 175 nm, or about 50 nm to about 150 nm. For embodiments that are unilamellar nanoparticles or liposomes, the first layer is also an outer layer of the nanoparticles. Liposomes can also be double bilayer vesicles or multilamellar or oligolamellar vesicles (in which the innermost layer is the first layer surrounding the core region), which generally have diameters in the range of about 300 nm or more, and about 500 nm or more, respectively, with concentric lipid bilayers alternating with layers of an aqueous phase. In some embodiments, the nanoparticle is a liposome and the liposome has a diameter of between about 50 nm and 150 nm, or between about 75 nm and about 125 nm, as measured by dynamic light scattering (DLS).

The lipids of the liposome can be cationic, zwitterionic, neutral or anionic, or any mixture thereof. Suitable lipids can include fats, waxes, steroids, cholesterol, fat-soluble vitamins, monoglycerides, diglycerides, phospholipids, sphingolipids, glycolipids, cationic or anionic lipids, derivatized lipids, and the like.

Suitable phospholipids include but are not limited to phosphatidylcholine (PC), phosphatidic acid (PA), phosphatidylethanolamine (PE), phosphatidylglycerol (PG), phosphatidylserine (PS), and phosphatidylinositol (PI), dimyristoyl phosphatidyl choline (DMPC), distearoyl phosphatidyl choline (DSPC), dioleoyl phosphatidyl choline (DOPC), dipalmitoyl phosphatidyl choline (DPPC), dimyristoyl phosphatidyl glycerol (DMPG), distearoyl phosphatidyl glycerol (DSPG), dioleoyl phosphatidyl glycerol (DOPG), dipalmitoyl phosphatidyl glycerol (DPPG), dimyristoyl phosphatidyl serine (DMPS), distearoyl phosphatidyl serine (DSPS), dioleoyl phosphatidyl serine (DOPS), dipalmitoyl phosphatidyl serine (DPPS), dioleoyl phosphatidyl ethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyp-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-0-monomethyl PE, 16-0-dimethyl PE, 18-1-trans PE, 1-stearoyl-2-oleoyl-phosphatidyethanolamine (SOPE), 1,2-dielaidoyl-sn-glycero-3-phophoethanolamine (transDOPE), and cardiolipin. Lipid extracts, such as egg PC, heart extract, brain extract, liver extract, and soy PC, are also useful in the present invention. In some embodiments, soy PC can include Hydro Soy PC (HSPC). In certain embodiments, the lipids can include derivatized lipids, such as PEGylated lipids. Derivatized lipids can include, for example, DSPE-PEG2000, cholesterol-PEG2000, DSPE-polyglycerol, or other derivatives generally known in the art.

Liposomes and lipid nanoparticles of the present disclosure may contain steroids, characterized by the presence of a fused, tetracyclic gonane ring system. Examples of steroids include, but are not limited to, cholesterol, cholic acid, progesterone, cortisone, aldosterone, estradiol, testosterone, dehydroepiandrosterone. Synthetic steroids and derivatives thereof are also contemplated for use in the present invention.

Cationic lipids contain positively charged functional groups under physiological conditions. Cationic lipids include, but are not limited to, N,N-dioleyl-N,N-dimethylammonium chloride (DODAC), N,N-distearyl-N,N-dimethylammonium bromide (DDA13), N-(1-(2,3-dioleoyloxy) propyl)-N,N,N-trimethylammonium chloride (DOTAP), N-(1-(2,3-dioleyloxy) propyl)-N,N,N-trimethylammonium chloride (DOTMA), N-[1-(2,3,-ditetradecyloxy) propyl1-N,N-dimethyl-N-hydroxyethylammonium bromide (DMRIE), N-[1-(2,3,dioleyloxy) propyl]-N,N-dimethyl-N-hydroxy ethylammonium bromide (DORIE), 3134N-(N′,N′-dimethylaminoethane) carbamoyl]cholesterol (DC-Chol), dimethyldioctadecylammonium (DDAB) and N,N-dimethyl-2,3-dioleyloxy)propylamine (DODMA).

Any suitable combination of lipids can be used to provide the liposomes and lipid-based nanoparticles of the disclosure. The lipid compositions can be tailored to affect characteristics such as leakage rates, stability, particle size, zeta potential, protein binding, in vivo circulation, and/or accumulation in tissues or organs. For example, DSPC and/or cholesterol can be used to decrease leakage from liposomes. Negatively or positively charged lipids, such as DSPG and/or DOTAP, can be included to affect the surface charge of a liposome or lipid-based nanoparticle. In some embodiments, the lipid compositions can include about ten or fewer types of lipids, or about five or fewer types of lipids, or about three or fewer types of lipids.

In some embodiments, the molar percentage or mole ratio of a specific type of lipid present typically comprises from about 0% to about 10% (or about 0 to about 10 for mole ratio), from about 10% to about 30% (about 0 to about 30 for mole ratio), from about 30% to about 50% (about 30 to about 50 for mole ratio), from about 50% to about 70% (about 50 to about 70 for mole ratio), from about 70% to about 90% (about 70 to about 90 for mole ratio), from about 90% to 100% (about 90 to about 100 for mole ratio) of the total lipid present in a liposome or lipid-based nanoparticle.

Any phosphatidylcholine (PC) or polyethylene glycol (PEG) may be used as appropriate for an intended use. In some embodiments, the nanoparticle includes a phosphatidylcholine (PC), cholesterol, a polyethylene glycol (PEG), in which the phosphatidylcholine includes one or more of phosphatidylcholine (PC), soy L-α-phosphatidylcholine (HSPC), dimyristoyl phosphatidyl choline (DMPC), distearoyl phosphatidyl choline (DSPC), dioleoyl phosphatidyl choline (DOPC), and dipalmitoyl phosphatidyl choline (DPPC); the cholesterol includes one or more of cholesterol and cholesterol-PEG2000; and the polyethylene glycol includes one or more of polyethylene glycol and 1,2-distearoylsn- glycero-3 phosphoethanolamine-N-[methoxy (polyethylene glycol)-2000](DSPE-PEG or DSPE-PEG-2000).

In some embodiments, the nanoparticle includes a phosphatidylcholine (PC), cholesterol, and a polyethylene glycol (PEG), in which the phosphatidylcholine is phosphatidylcholine and/or soy L-α-phosphatidylcholine (HSPC), and the polyethylene glycol is one or more of polyethylene glycol and DSPE-PEG. In other embodiments, the nanoparticle includes HSPC, cholesterol, and DSPE-PEG.

In some embodiments, the molar ratio of a phosphatidylcholine (PC) to a cholesterol in the liposome is between about 6:1 and about 2:1. In some embodiments, the molar ratio of a phosphatidylcholine (PC) to a cholesterol in the liposome is between about 5:1 and about 3:1, while in some embodiments, the molar ratio of a phosphatidylcholine (PC) to a cholesterol in the liposome is about 3.25:1, or about 3.5:1, or about 3.75:1, or about 3.8:1, or about 3.9:1, or about 4:1, or about 4.2:1, or about 4.3:1, or about 4.4:1, or about 4.5:1, or about 4.6:1, or about 4.7:1, or about 4.8:1, or about 4.9:1. In some embodiments, the molar ratio of a phosphatidylcholine (PC) to a cholesterol in the liposome is about 77:20 (or about 3.85:1).

In some embodiments, the molar ratio of a cholesterol to a polyethylene glycol (PEG) in the liposome is between about 9:1 and about 5:1. In some embodiments, the molar ratio of a phosphatidylcholine (PC) to a cholesterol in the liposome is between about 8:1 and about 6:1, while in some embodiments, the molar ratio of a cholesterol to a polyethylene glycol (PEG) in the liposome is about 20:3.2, or about 20:3.1, or about 20:3.05, or about 20:2.95, or about 20:2.9, about 20:2.8, or about 20:2.7, or about 20:2.6. In some embodiments, the molar ratio is about 20.3.

In some embodiments, the molar ratio of a phosphatidylcholine (PC) to a cholesterol to a polyethylene glycol (PEG) in the liposome is about 77:20:3, or about 78:20:2, or about 78:19:3, or about 78:18:4, or about 78:21:1, or about 79:18:3, or about 79:19:2, or about 79:17:4, or about 80:17:3, or about 80:18:2, or about 80:19:1, or about 80:16:4, or about 77:19:4, or about 77:18:5, or about 77:21:2, or about 77:22:1, or about 76:21:3, or about 76:22:2, or about 76:23:1, or about 76:20:4, or about 76:19:5, and in some embodiments, the phosphatidylcholine is HSPC and the PEG is DSPE-PEG.

When forming a lipid-based nanoparticle that includes a PEG, the PEG molecules may be present on the outer surface of the liposome or the inner surface of the liposome, lining the core region. In some embodiments, a majority (i.e., more than 50%) of the PEG is present on the liposome surface. In some embodiments, the PEG is one or more of polyethylene glycol and DSPE-PEG, and in some embodiments, the PEG is DSPE-PEG.

In some embodiments, the nanoparticle is a polymer-based nanoparticle. Polymer-based nanoparticles that possess a core region surrounded by a polymeric shell are also referred to as nanocapsules.

Any polymer can be used in accordance with the present invention. Polymers can be natural or unnatural (synthetic) polymers. Polymers can be homopolymers or copolymers comprising two or more monomers. In terms of sequence, copolymers can be random, block, or comprise a combination of random and block sequences.

The term “polymer,” as used herein, is given its ordinary meaning as used in the art, i.e., a molecular structure comprising one or more repeat units (monomers), connected by covalent bonds. The repeat units may all be identical, or in some cases, there may be more than one type of repeat unit present within the polymer. In some cases, the polymer can be biologically derived, i.e., a biopolymer. Non-limiting examples include peptides or proteins. In some cases, additional moieties may also be present in the polymer, for example biological moieties such as those described below. If more than one type of repeat unit is present within the polymer, then the polymer is said to be a “copolymer.” It is to be understood that in any embodiment employing a polymer, the polymer being employed may be a copolymer in some cases. The repeat units forming the copolymer may be arranged in any fashion. For example, the repeat units may be arranged in a random order, in an alternating order, or as a block copolymer, i.e., comprising one or more regions each comprising a first repeat unit (e.g., a first block), and one or more regions each comprising a second repeat unit (e.g., a second block), etc. Block copolymers may have two (a diblock copolymer), three (a triblock copolymer), or more numbers of distinct blocks.

Disclosed particles can include copolymers, which, in some embodiments, describes two or more polymers (such as those described herein) that have been associated with each other, usually by covalent bonding of the two or more polymers together. Thus, a copolymer may include a first polymer and a second polymer, which have been conjugated together to form a block copolymer where the first polymer can be a first block of the block copolymer and the second polymer can be a second block of the block copolymer. Of course, those of ordinary skill in the art will understand that a block copolymer may, in some cases, contain multiple blocks of polymer, and that a “block copolymer,” as used herein, is not limited to only block copolymers having only a single first block and a single second block. For instance, a block copolymer may include a first block including a first polymer, a second block including a second polymer, and a third block including a third polymer or the first polymer, etc. In some cases, block copolymers can contain any number of first blocks of a first polymer and second blocks of a second polymer (and in certain cases, third blocks, fourth blocks, etc.). In addition, it should be noted that block copolymers can also be formed, in some instances, from other block copolymers. For example, a first block copolymer may be conjugated to another polymer (which may be a homopolymer, a biopolymer, another block copolymer, etc.), to form a new block copolymer containing multiple types of blocks, and/or to other moieties (e.g., to non-polymeric moieties).

In some embodiments, the polymer (e.g., copolymer, e.g., block copolymer) can be amphiphilic, i.e., having a hydrophilic portion and a hydrophobic portion, or a relatively hydrophilic portion and a relatively hydrophobic portion. A hydrophilic polymer can be one generally that attracts water and a hydrophobic polymer can be one that generally repels water. A hydrophilic or a hydrophobic polymer can be identified, for example, by preparing a sample of the polymer and measuring its contact angle with water (typically, the polymer will have a contact angle of less than 60°, while a hydrophobic polymer will have a contact angle of greater than about) 60°. In some cases, the hydrophilicity of two or more polymers may be measured relative to each other, i.e., a first polymer may be more hydrophilic than a second polymer. For instance, the first polymer may have a smaller contact angle than the second polymer.

In one set of embodiments, a polymer (e.g., copolymer, e.g., block copolymer) contemplated herein includes a biocompatible polymer, i.e., a polymer that does not typically induce an adverse response when inserted or injected into a living subject, such as significant inflammation and/or acute rejection of the polymer by the immune system (e.g., via a T-cell response). Accordingly, polymer-based nanoparticles described herein can be non-immunogenic.

One test to determine biocompatibility can be to expose a polymer to cells in vitro; biocompatible polymers are polymers that typically do not result in significant cell death at moderate concentrations, e.g., at concentrations of 50 micrograms/10⁶ cells. For instance, a biocompatible polymer may cause less than about 20% cell death when exposed to cells such as fibroblasts or epithelial cells, even if phagocytosed or otherwise taken up by such cells. Non-limiting examples of biocompatible polymers that may be useful in various embodiments include polydioxanone (PDO), polyhydroxyalkanoate, polyhydroxybutyrate, poly(glycerol sebacate), polyglycolide, polylactide, PLGA, polycaprolactone, or copolymers or derivatives including these and/or other polymers.

In some embodiments, contemplated biocompatible polymers may be biodegradable, i.e., the polymer is able to degrade, chemically and/or biologically, within a physiological environment, such as within the body. As used herein, “biodegradable” polymers are those that, when introduced into cells, are broken down by the cellular machinery (biologically degradable) and/or by a chemical process, such as hydrolysis, (chemically degradable) into components that the cells can either reuse or dispose of without significant toxic effect on the cells. In one embodiment, the biodegradable polymer and their degradation byproducts can be biocompatible.

For instance, a contemplated polymer may be one that hydrolyzes spontaneously upon exposure to water (e.g., within a subject), the polymer may degrade upon exposure to heat (e.g., at temperatures of about 37° C. or more). Degradation of a polymer may occur at varying rates, depending on the polymer or copolymer used. For example, the half-life of the polymer (the time at which 50% of the polymer can be degraded into monomers and/or other nonpolymeric moieties) may be on the order of days, weeks, months, or years, depending on the polymer. The polymers may be biologically degraded, e.g., by enzymatic activity or cellular machinery, in some cases, for example, through exposure to a lysozyme (e.g., having relatively low pH). In some cases, the polymers may be broken down into monomers and/or other nonpolymeric moieties that cells can either reuse or dispose of without significant toxic effect on the cells (for example, polylactide may be hydrolyzed to form lactic acid, polyglycolide may be hydrolyzed to form glycolic acid, etc.).

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEGylated polymers and copolymers of lactide and glycolide (e.g., PEGylated PLA, PEGylated PGA, PEGylated PLGA, and derivatives thereof. In some embodiments, polyesters include, for example, polyanhydrides, poly(ortho ester) PEGylated poly(ortho ester), poly(caprolactone), PEGylated poly(caprolactone), polylysine, PEGylated polylysine, poly(ethylene imine), PEGylated poly(ethylene imine), poly(L-lactide-co-L-lysine), poly(serine ester), poly(4-hydroxy-L-proline ester), poly[α-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA can be characterized by the ratio of lactic acid:glycolic acid. Lactic acid can be L-lactic acid, D-lactic acid, or D,L-lactic acid. The degradation rate of PLGA can be adjusted by altering the lactic acid-glycolic acid ratio. In some embodiments, PLGA to be used in accordance with the present invention can be characterized by a lactic acid:glycolic acid ratio of approximately 85:15, approximately 75:25, approximately 60:40, approximately 50:50, approximately 40:60, approximately 25:75, or approximately 15:85. In some embodiments, the ratio of lactic acid to glycolic acid monomers in the polymer of the particle (e.g., the PLGA block copolymer or PLGA-PEG block copolymer), may be selected to optimize for various parameters such as water uptake, therapeutic agent release and/or polymer degradation kinetics can be optimized.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), methacrylic acid alkylamide copolymer, poly(methyl methacrylate), poly(methacrylic acid polyacrylamide, amino alkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids (e.g. DNA, RNA, or derivatives thereof). Amine-containing polymers such as poly(lysine), polyethylene imine (PEI), and poly(amidoamine) dendrimers are contemplated for use, in some embodiments, in a disclosed particle.

In some embodiments, polymers can be degradable polyesters bearing cationic side chains. Examples of these polyesters include poly(L-lactide-co-L-lysine), poly(serine ester), poly(4-hydroxy-L-proline ester).

In some embodiments, the nanoparticle includes a mixture of one or more lipids and one or more polymers.

Adding one or more particle modifiers to the first layer of the nanoparticles can be used to tune particle morphology, including but not limited to adjusting the loading amount, altering the release kinetics and the transport characteristics of a molecule present, e.g., a therapeutic agent, in the core region. The particle modifier may adjust or temporarily adjust one or more particle layers that impact the molecular partition rates and lipophilicity of the particle. Particle modifiers include compounds, including but not limited to excipients, therapeutic agents, vaccine agents, and the like, that can modify one or more properties of the nanoparticle.

In some embodiments, the one or more particle modifiers includes a therapeutic or therapeutic agent. In embodiments, the one or more therapeutics can include an anticancer agent or cytotoxic agent including but not limited to avastin, doxorubicin, cisplatin, oxaliplatin, carboplatin, 5-fluorouracil, gemcitibine or taxanes, such as paclitaxel and docetaxel. Additional anti-cancer agents can include but are not limited to 20-epi-1,25 dihydroxyvitamin D3,4-ipomeanol, 5-ethynyluracil, 9-dihydrotaxol, abiraterone, acivicin, aclarubicin, acodazole hydrochloride, acronine, acylfulvene, adecypenol, adozelesin, aldesleukin, all-tk antagonists, altretamine, ambamustine, ambomycin, ametantrone acetate, amidox, amifostine, aminoglutethhnide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisaziridinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caracemide, carbetimer, carboplatin, carboxamide-amino-triazole, carboxyamidotriazole, carest M3, carmustine, cam 700, cartilage derived inhibitor, carubicin hydrochloride, carzelesin, casein kinase inhibitors, castanospermine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethylnorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocannycin SA, ebselen, ecomustine, edatrexate, edelfosine, edrecolomab, eflomithine, eflomithine hydrochloride, elemene, elsamitrucin, emitefur, enloplatin, enprornate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, fluorocitabine, forfenimex, fonnestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ilmofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatin, irinotecan, irinotecan hydrochloride, iroplact, irsogladine, isobengazole, isohornohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide 7, lobaplatin, lombricine, lometrexol, lometrexol sodium, lomustine, lonidamine, losoxantrone, losoxantrone hydrochloride, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor I-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, 06-benzylguanine, octreotide, okicenone, oligonucleotides, onapristone, ondansetron, oracin, oral cytokine inducer, orinaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RIT retinamide, RNAi, rogletimide, rohitukine, romurtide, roquinimex, rubiginone B 1, ruboxyl, safingol, safingol hydrochloride, saintopin, sarcnu, sarcophytol A, sargramostim, SDI 1 mimetics, semustine, senescence derived inhibitor 1, sense oligonucleotides, signal transduction inhibitors, signal transduction modulators, simtrazene, single chain antigen binding protein, sizofuran, sobuzoxane, sodium borocaptate, sodium phenylacetate, solverol, somatomedin binding protein, sonermin, sparfosate sodium, sparfosic acid, sparsomycin, spicamycin D, spirogermanium hydrochloride, spiromustine, spiroplatin, splenopentin, spongistatin 1, squalamine, stem cell inhibitor, stern-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, tcloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zinostatin stimalamer, or zorubicin hydrochloride, or pharmaceutically acceptable salts thereof.

A therapeutic or therapeutic agent or combination therapy of two or more therapeutics can be selected depending on the type of disease to be treated. For example, many types of cancers or tumors exhibit different responses, e.g., a lesser response, a greater response or no response, to different therapeutics. Thus, in certain embodiments, therapeutics for incorporation in nanoparticles, either as one or more particle modifiers incorporated within, present in or present on the first layer, or as one or more therapeutics present in the core region, or both, are selected that are effective against the specific disease being treated and compatible with the physical characteristics of the first layer (e.g., polymer-based or lipid-based) and the core region (e.g., aqueous).

Nucleic acids can also act as therapeutics. The disclosure therefore provides for the incorporation of one or more nucleic acids in a nanoparticle, e.g., in the core region, in addition to the one or more particle modifiers added to the first layer of the nanoparticle. Release of nucleic acids from a nanoparticle of the disclosure, such as a mRNA, or a siRNA or microRNA can lead to a target protein production or protein silencing, respectively; both mechanisms of which can be exploited to lead to cell death in anti-cancer applications. The combination of a nucleic acid and non-nucleic acid therapeutic in a single nanoparticle is another type of a multi-drug loaded particle. As a non-limiting example, docetaxel and mRNA can be loaded simultaneously into a lipid-based particle, or any other lipid/polymer particle, where the ratios of docetaxel (as a particle modifier and therapeutic) to other first layer (e.g., lipid bilayer) components such can be used to further tune the morphology of the particle. In some embodiments, the one or more therapeutic present in the core region comprises a nucleic acid, including but not limited to an RNA such as a mRNA, microRNA, a siRNA and/or shRNA.

In some embodiments, the nanoparticle is a liposome and the one or more therapeutics present in the core region is present in an aqueous core of the liposome.

In some embodiments, the particle modifier includes one or more poorly water-soluble second therapeutics, which, in some embodiments includes a taxane. In some embodiments, the taxane comprises docetaxel (DTX), or a pharmaceutically acceptable salt thereof. In some embodiments, the one or more therapeutics present in the core region comprises an amphipathic weak acid or base.

In some embodiments that include one or more lipids (e.g., liposomes), the one or more particle modifiers is present in the first layer that surrounds the core region (a lipid bilayer (lamella) of the liposome). Such particle modifiers may reside completely within the lipid bilayer not exposed to solvent on either side of the bilayer, or intercalate within the lipids such that at least a part of the particle modifier is exposed to solvent.

In some embodiments, the one or more therapeutics present in the core region comprise an amphipathic weak acid or base. In other embodiments, the particle modifier includes one or more poorly water-soluble second therapeutics, which, in some embodiments includes a taxane generally, or docetaxel (DTX), and the one or more therapeutics present in the core region comprise an amphipathic weak acid or base, which in some embodiments includes doxorubicin (DOX).

In some embodiments, the nanoparticle is a liposome that includes a phospholipid bilayer surrounding an aqueous core, docetaxel (DTX), or a salt thereof, as a particle modifier present in a phospholipid bilayer of the liposome, and doxorubicin (DOX), or a salt thereof, present in the core region of the nanoparticle, which core region is aqueous. The docetaxel acts as a second therapeutic, and in some embodiments, a structural modifier as described elsewhere herein.

In some embodiments, the one or more therapeutics present in the core region is selected from the group consisting of doxorubicin, daunorubicin, epirubicin, idarubicin, vincristine and irinotecan hydrochloride, or pharmaceutically acceptable salts thereof.

In some embodiments, and as described in the examples, docetaxel can be added to a liposomal bilayer as a particle modifier, where the docetaxel acts as both an active ingredient (a therapeutic) and as a particle modifier in the nanoparticle bilayer, adjusting one or more properties of the particle, such as altering the partition coefficient of the loading molecule, which would tune the permeation through the outer layer of the nanoparticle. This modifier can then promote the drug loading or encapsulation of a second molecule including but not limited to amphipathic weak acids and bases such as doxorubicin, daunorubicin, epirubicin, idarubicin, vincristine and irinotecan hydrochloride. These amphipathic molecules may be loaded into the particles, for example, via an active loading method.

In some embodiments, nanoparticles as described in various aspects and embodiments herein (i.e., including, e.g., liposomes), include one or more structural modifiers (including therapeutic like DTX or a salt thereof), present at a concentration of between about 0.1 mg/ml and about 0.75 mg/ml, or between about 0.2 mg/ml and about 0.6 mg/ml, or between about 0.25 mg/ml and about 0.5 mg/ml, or between about 0.3 nm/ml and 0.4 mg/ml.

In some embodiments, nanoparticles as described in various aspects and embodiments herein include one or more therapeutics present in the core region (e.g., DOX or a salt thereof) at a concentration of between about 0.25 mg/ml and about 2 mg/ml, or between about 0.5 mg/ml and about 1.75 mg/ml, or between about 0.75 mg/ml and about 1.25 mg/ml, or between about 0.9 nm/ml and 1.1 mg/ml.

In some embodiments, nanoparticles as described in various aspects and embodiments herein include one or more structural modifiers (including therapeutic like DTX or a salt thereof), present at a concentration of between about 0.1 mg/ml and about 0.75 mg/ml, or between about 0.2 mg/ml and about 0.6 mg/ml, or between about 0.25 mg/ml and about 0.5 mg/ml, or between about 0.3 nm/ml and 0.4 mg/ml, as well as one or more therapeutics present in the core region (e.g., DOX or a salt thereof) at a concentration of between about 0.25 mg/ml and about 2 mg/ml, or between about 0.5 mg/ml and about 1.75 mg/ml, or between about 0.75 mg/ml and about 1.25 mg/ml, or between about 0.9 nm/ml and 1.1 mg/ml.

The addition of a particle modifier can alter the nanostructure (e.g. a nanocrystal) of one or more therapeutics once it is inside the core region of the nanoparticle. As further described in the examples and elsewhere herein, doxorubicin nanocrystals can form in an elongated crystal shape, as opposed to a circular crystal shape, or an elongated nanoparticle versus a spherical nanoparticle. Different particle modifiers and amount (or ratio) of modifier in a liposomal bilayer can be used to adjust the particle morphology and pharmacokinetic/ pharmacodynamic characteristics of a formulation. Thus, in some embodiments, the nanoparticle is a liposome in which one or more therapeutics (in some embodiments including DOX or salt thereof) is present in the core region of the nanoparticle, and one or more of the therapeutics present in the core region (including but not limited to an aqueous core of a liposome) comprises crystals or is in a crystalized form. One or more therapeutics present in the core region can comprise non-crystalized therapeutic, and in some embodiments, both crystallized and non-crystallized one or more therapeutics is present in the core region. In some embodiment, a majority (more than 50%) of the one or more therapeutic present in the core region (in some embodiments including DOX in an aqueous core) is crystalized. In other embodiments, at least 75%, 80%, 85%, 90%, 95%, or 100% of the one or more therapeutic present in the core region (in some embodiments including DOX in an aqueous core) is crystalized.

In some embodiments, the nanoparticle is a liposome and the liposome has an aspect ratio of between about 1 and about 4, or between about 1 and about 3, or between about 1 and about 2.5, or between about 1 and about 2, or between about 1 and about 1.75, or between about 1 and about 1.6, or between about 1 and about 1.4, where the aspect ratio is calculated by dividing the major axis by the minor axis.

As described in the examples and elsewhere herein, for docetaxel and doxorubicin, both molecules are therapeutics, and the combination of these molecules in a single particle can increase therapeutic efficacy while reducing the total amount of active ingredients needed to be effective, which can reduce clinical side effects such as tissue toxicity (e.g. cardiotoxicity). Moreover, the multi-drug approach enables multiple molecular pathways to be triggered within a cancerous cell. For anti-cancer applications, triggering more than one pathway that leads to cellular death can greatly enhance the overall efficacy of a drug product, such as a nanoparticle as described herein.

As shown in the non-limiting examples, novel docetaxel (DTX) and doxorubicin (DOX) co-encapsulated liposomes are disclosed, manufactured using a continuous process. Stable liposomes were first formed with DTX in the lipid bilayer and then DOX was actively loaded into the inner aqueous compartment of the particle. This was accomplished without the loss of the first drug. The co-encapsulated formulation was stable for the duration of the testing period (3 months) and showed significantly (p<0.05) higher cytotoxicity in multiple cell lines than the free drug and also the single-therapeutic liposomal formulations (given individually or in combination). Continuous manufacturing enabled the rapid development and production of a novel co-encapsulated liposomes, providing pharmacokinetic and therapeutic advantages.

In another aspect of the disclosure, a method for making nanoparticles as described herein is provided, the method including dissolving lipid components and particle modifier (e.g., second therapeutic, including DTX or a salt thereof), in an organic solvent to produce a lipid-particle modifier-organic solvent solution; injecting the lipid-particle modifier-organic solvent solution into a larger volume of an aqueous solution, forming a turbulent jet at the site where the lipid-organic solvent solution and the aqueous solution mix, forming liposomes; concentrating the liposomes; and mixing the concentrated liposomes with a solution of a first therapeutic or a salt thereof, at an elevated temperature sufficient to enable loading of the one or more first therapeutic or a salt thereof. Suitable temperatures can include those of about 45° C. or higher, or about 45° C., or about 50° C. or higher, or about 50° C., about 55° C. or higher, or about 55° C., about 60° C. or higher, or about 60° C., about 65° C. or higher, or about 65° C., 70° C. or higher, or at about 70° C., about 75° C. or higher, or about 75° C., about 80° C. or higher, or about 80° C., about 85° C. or higher, or about 85° C., about 90° C. or higher, or about 90° C., or between about 50° C. and about 90° C., and interim ranges therein as described above, including between about 57° C. and 83° C., or between about 62° C. and 78° C., or between about 65° C. and 75° C., or between about 61° C. and 74° C., or between about 68° C. and 74° C.

Any suitable techniques and devices for mixing the solvent and aqueous solutions, and concentrating liposomes, etc., may be used in conjunction with the methods of the disclosure. Exemplary and non-limiting such methods are described herein and in U.S. Pat. No. 10632072, incorporated by reference herein in its entirety.

Initial steps can be done at any suitable temperature, as described herein, or, in one non-limiting embodiment, at about room temperature. Suitable organic solvents include, e.g., ether, ethanol, methanol, isopropanol, DMSO. The aqueous solution can comprise water or a buffer. Where the aqueous solution is a buffer, it can comprise pharmaceutically acceptable excipients, buffers, salts, acids, bases and/or sugars (e.g., compositions comprising saline or phosphate buffered saline (PBS)). Sugars, such as sucrose, cyclodextran and natural polysaccharides can be used in the formulation of aqueous solutions. In certain embodiments, a pharmaceutically acceptable excipient is added as an aqueous solution.

In some embodiments, the method further includes removing the one or more therapeutics present in the core region that are not encapsulated in liposomes.

As for the nanoparticles described elsewhere herein, in some embodiments of the method for making nanoparticles, the first therapeutic comprises an amphipathic weak acid or base, which, in certain embodiments is selected from the group consisting of doxorubicin, daunorubicin, epirubicin, idarubicin, vincristine and irinotecan hydrochloride, or pharmaceutically acceptable salts thereof. In some embodiments, the first therapeutic includes a nucleic acid, including but not limited to an RNA such as a mRNA, microRNA, an siRNA or a shRNA.

Also as for the nanoparticles described elsewhere herein, in some embodiments of the method for making nanoparticles, the lipid components comprise a PC and cholesterol. In some embodiments, the PC includes HSPC. In embodiments of the method, the nanoparticle includes a molar ratio of a PC:a cholesterol, or HSPC:a cholesterol in a lipid-DTX-organic solvent solution of between about 5:1 and about 3:1, or of about 77:20.

In some embodiments of the method for making nanoparticles, the concentration of lipid components in the lipid-particle modifier-organic solvent solution is about 2.5 nM to about 25 nM, about 5 nM to about 15 mM, about 7.5 mM to about 12.5 mM, or about 10 mM.

In some embodiments, the lipid-particle modifier-organic solvent solution further comprises PEG, which, in some embodiments, comprises DSPE-PEG.

Also as for the nanoparticles described elsewhere herein, in some embodiments of the method for making nanoparticles, the molar ratio of a PC:a cholesterol:a PEG, or a molar ratio of HSPC:a cholesterol:DSPE-PEG in the lipid-particle modifier-organic solvent solution is about 77:20:3, or about 78:20:2, or about 78:19:3, or about 78:18:4, or about 78:21:1, or about 79:18:3, or about 79:19:2, or about 79:17:4, or about 80:17:3, or about 80:18:2, or about 80:19:1, or about 80:16:4, or about 77:19:4, or about 77:18:5, or about 77:21:2, or about 77:22:1, or about 76:21:3, or about 76:22:2, or about 76:23:1, or about 76:20:4, or about 76:19:5. In some embodiments, the the molar ratio of a PC:a cholesterol:a PEG, or a molar ratio of HSPC:a cholesterol:DSPE-PEG in the lipid-particle modifier-organic solvent solution is about 77:20:3.

In some embodiments of the method for making nanoparticles, the organic solvent includes one or more of ether, ethanol, methanol, isopropanol, and DMSO, and in other embodiment, the organic solvent is ethanol.

The aqueous solution described in the method for making nanoparticles as described herein can include water or any suitable buffer. In some embodiments, the aqueous solution includes about 300 mM ammonium sulfate.

Therapeutics for incorporation into the core region of nanoparticles of the disclosure can be dissolved in any suitable solution. Suitable solutions can include buffers and other compounds to, e.g., increase dissolution, increase stability, facilitate therapeutic transfer to the core region and the like. In some embodiments of the method for making nanoparticles as described herein can include a histidine buffer, and in some embodiments, the histidine buffer is at a concentration of about 20 mM and at a pH of about 6.0. In some embodiments, the solution further comprises a PEG, which, in some embodiments, is polyethylene glycol, and in some embodiments, DSPE-PEG.

In another aspect, the disclosure provides nanospheres made according to the method for making nanoparticles as described herein.

Pharmaceutical Formulations

Nanoparticles disclosed herein may be combined with pharmaceutical acceptable carriers to form a pharmaceutical composition, according to another aspect of the invention. Typically, the physiologically acceptable carriers are present in liquid form. Examples of liquid carriers include physiological saline, phosphate buffer, normal buffered saline (135-150 mM NaCl), water, buffered water, 0.4% saline, 0.3% glycine, glycoproteins to provide enhanced stability (e.g., albumin, lipoprotein, globulin, etc.), and the like. Since physiologically acceptable carriers may be chosen, e.g., based on the route of administration as described below, the location of the target issue, the drug being delivered, the time course of delivery of the drug, and the like, there are a wide variety of suitable formulations of pharmaceutical compositions for embodiments of the present disclosure (See, e.g., Remington's Pharmaceutical Sciences, 17th ed., 1989).

The pharmaceutical compositions of this invention can be administered to a patient by any means known in the art, including oral and parenteral routes. In certain embodiments parenteral routes are desirable since they avoid contact with the digestive enzymes that are found in the alimentary canal. According to such embodiments, inventive compositions may be administered by injection (e.g., intravenous, subcutaneous or intramuscular, intraperitoneal injection), rectally, vaginally, topically (as by powders, creams, ointments, or drops), or by inhalation (as by sprays).

In a particular embodiment, the nanoparticles of the present invention are administered to a subject in need thereof systemically, e.g., by IV infusion or injection.

Injectable preparations, for example, sterile injectable aqueous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed include water, Ringer's solution, U.S.P., and isotonic sodium chloride solution. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the encapsulated or unencapsulated conjugate is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

It will be appreciated that the exact dosage of nanoparticle of the disclosure is chosen by the individual physician in view of the patient to be treated. Typically, dosage and administration are adjusted to provide an effective amount of the nanoparticles to the patient being treated. As will be appreciated by those of ordinary skill in this art, the effective amount of nanoparticle may vary depending on such factors as the desired biological endpoint, the therapeutic(s) to be delivered, the target tissue, the route of administration, and the like. For example, the effective amount of nanoparticles containing a particle modifier that is also an anti-cancer therapeutic, as well as a second (or more) therapeutic in the core region, might be the amount that results in a reduction in tumor size by a desired amount over a desired period of time. Additional factors which may be taken into account include the severity of the disease state; age, weight and gender of the patient being treated; diet, time and frequency of administration; drug combinations; reaction sensitivities; and tolerance/response to therapy.

The nanoparticles of the disclosure may be formulated in unit dosage form for ease of administration and uniformity of dosage. The expression “unit dosage form” as used herein refers to a physically discrete unit of nanoparticle appropriate for the patient to be treated. It will be understood, however, that the total daily usage of the compositions of the present disclosure will be decided by the attending physician within the scope of sound medical judgment. For any nanoparticle or nanoparticle composition, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. Therapeutic efficacy and toxicity of nanoparticles can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies can be used in formulating a range of dosage for human use.

In some embodiments, a composition is provided including a plurality of nanoparticles of the various embodiments described herein, and one or more of a pharmaceutically acceptable carrier, a pharmaceutically acceptable excipient, or pharmaceutically acceptable diluent. Such compositions can further comprise any other components as appropriate for an intended use of the nanoparticle composition.

In some embodiments of the composition, the plurality of nanoparticles or liposomes have a mean particle diameter of between about 80 nm and about 120 nm, or between about 90 nm and about 110 nm, or between about 95 nm and 105 nm, as measured by dynamic light scattering (DLS). In some embodiments of the composition, the plurality of nanoparticles or liposomes have a polydispersity index of less than or equal to 0.1, as measured by dynamic light scattering (DLS), and in some embodiments, the plurality of nanoparticles or liposomes have a zeta potential of between about −15 mV to about −50 mV, as measured by dynamic light scattering (DLS).

In some embodiments, a composition suitable for freezing is provided, including nanoparticles as disclosed herein and a solution suitable for freezing, e.g. a sucrose solution is added to the nanoparticle suspension. The sucrose may act, e.g., as a cryoprotectant to prevent the particles from aggregating upon freezing. For example, provided herein is a nanoparticle formulation comprising a plurality of disclosed nanoparticles, sucrose and water; wherein the nanoparticles/sucrose/water is about 3-30%/10-30%/50-90% (w/w/w) or about 5-10%/10-15%/80-90% (w/w/w).

Methods of Treatment

In an aspect of the disclosure, nanoparticles as described in various aspects and embodiments herein can be used to treat a disease, disorder, and/or condition. In some embodiments, nanoparticles of the disclosure, or compositions thereof, may be used to treat solid tumors, e.g., cancer and/or cancer cells. The methods provided herein may be used to treat any cancer type as deemed appropriate by attending medical personnel.

In some embodiments, a method for the treatment of cancer (e.g., prostate, breast cancer, and the like) is provided. In some embodiments, the treatment of cancer includes administering a therapeutically effective amount of the nanoparticles described herein, or composition thereof, to a subject having a cancer. Nanoparticles, or compositions thereof, can be administered in such amounts and for such time as is necessary to achieve the desired result as defined elsewhere herein but, in brief, treating, alleviating, ameliorating, relieving, delaying onset of, inhibiting progression or growth of, inhibiting the migration of, reducing severity of, and/or reducing incidence of one or more symptoms or features of cancer.

In some embodiments, a method for preventing cancer is provided. Such methods include administering a therapeutically effective amount of a nanoparticle or composition thereof to a healthy individual (i.e., a subject who does not display any symptoms of cancer and/or who has not been diagnosed with cancer). For example, healthy individuals may be “immunized” with an inventive targeted particle prior to development of cancer and/or onset of symptoms of cancer; at risk individuals (e.g., patients who have a family history of cancer; patients carrying one or more genetic mutations associated with development of cancer; patients having a genetic polymorphism associated with development of cancer; patients infected by a virus associated with development of cancer; patients with habits and/or lifestyles associated with development of cancer; etc.) can be treated prior to or substantially contemporaneously with (e.g., within 48 hours, within 24 hours, or within 12 hours of) the onset of symptoms of cancer.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein. No functional language used in claims appended herein is to be construed as invoking 35 U.S.C. § 112(f) interpretations as “means-plus-function” language unless specifically expressed as such by use of the words “means for” or “steps for” within the respective claim.

When introducing elements of the present invention or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. The term “exemplary” is not intended to be construed as a superlative example but merely one of many possible examples.

The following examples further illustrate the present invention but should not be construed as in any way limiting its scope.

### EXAMPLES

Anthracyclines and taxanes are two of the most widely used classes of anticancer therapeutics to treat several different cancers. Doxorubicin (DOX) was one of the first anthracycline chemotherapeutics and works by inhibiting cell growth by impeding the topoisomerase II enzyme activity. Docetaxel (DTX) belongs to the taxane family, which cause cell death by blocking the cell cycle at the G2/M phase through the inhibition of microtubule disassembly. Additionally, it has been shown that DTX is safer and more effective than paclitaxel despite both drugs utilizing the same method of action. Both DTX and DOX are effective anticancer therapeutics with two very different mechanisms and do not have overlapping toxicity profiles. The use of DTX is limited in clinical formulations due to its poor water solubility and relatively poor pharmacokinetics.

In the following examples, a novel docetaxel (DTX) and doxorubicin (DOX) co-encapsulated liposomal formulation was manufactured using a continuous process. Stable liposomes were first formed with DTX, and then DOX was actively loaded into the inner aqueous compartment. This was accomplished without the loss of the first therapeutic, DTX. The formulation displayed desirable physicochemical properties such as a monodispersed population and high encapsulation efficiency for both APIs. A mean diameter of 97.6±2.2 nm was observed, and a polydispersity index of 0.065 was achieved. The encapsulation efficacy (%) for DTX and DOX was 80.2 and 97.1, respectively. The co-encapsulated formulation was stable for the duration of the testing period (3 months) and showed significantly higher cytotoxicity (p<0.05) in multiple cell lines than the free drug and also the single-therapeutic liposomal formulations (given individually or in combination). The release profiles of the multidrug-loaded formulation indicated that the presence of one API influenced the release of the other, which is useful when designing a drug delivery system with staggered release kinetics. Continuous manufacturing methods enabled rapid production of the novel co-encapsulated liposomal formulation.

### Example 1: Liposome Preparation

The lipid components, including HSPC, cholesterol, and DSPE-PEG2000, were dissolved in ethanol at a molar ratio of 7.7:2:0.1, respectively. The total lipid concentration was 10 mM. The liposome formulations were manufactured using a continuous processing system based on an ethanol injection method. Briefly, an ethanolic solution containing the lipid components was rapidly injected through a needle into a larger volume of an aqueous medium. The co-flow technology employs the formation of a turbulent jet at the site where the two flows mix, promoting vesicle formation. Ammonium sulfate (300 mM) was used as the aqueous medium during liposome formation, and the amount of ethanol was diluted according to ICH guidelines for residual solvents. The liposomal formulations were then concentrated and buffer exchanged in-line using two consecutive tangential flow filtration (TFF) systems operating in the single-pass mode.

For formulations containing DTX, the active pharmaceutical ingredient (API) was passively loaded into the liposomes by dissolving in ethanol with the other lipid components prior to the formation of liposomes. For the formulation containing DTX and DOX, the DTX was passively loaded as previously mentioned, and the DOX was actively loaded in-line via remote loading driven by a transmembrane gradient. More specifically, the concentrated liposomes were mixed with a solution of DOX dissolved in histidine buffer (20 mM, pH 6.0) through a series of static mixers at an elevated temperature (70° C.). Any unencapsulated DOX was removed using a third TFF system containing single-pass capsule filters and then chilled to room temperature. DSPE-PEG2000 was also dissolved in the histidine buffer to incorporate onto the liposomes through the post-insertion process. All formulations contained a total of 3 mole percent of DSPE-PEG2000 and were passed through an in-line 0.22 μm filter prior to final collection in a 2D bag. All the formulations that were manufactured are listed in Table 1.

TABLE 1

| | Final Molar Ratio | | |
|---|---|---|---|
| Formulation | (HSPC:Cholesterol:DSPE- | [DTX] | [DOX] |
| ID | PEG2000) | (mg/mL) | (mg/mL) |
| Blank Lip | 77:20:3 | — | — |
| Lip-DTX | 77:20:3 | 0.325 | — |
| Lip-DOX | 77:20:3 | — | 1 |
| Lip-DTX/DOX | 77:20:3 | 0.325 | 1 |

For Table 1, “Blank Lip” represents liposomes containing no API; “Lip-DTX” represents liposomes containing only DTX; “Lip-DOX” represents liposomes containing only DOX; and “Lip-DTX/DOX” represents liposomes containing both DTX and DOX.

Physicochemical Characterization of Liposomal Formulations

### Example 2: Formulation Characterization—Determination of Particle Size Distribution and Zeta Potential

The particle size distribution (Z-average (d. nm)), polydispersity index (PDI), and zeta potential (mV) were measured offline using dynamic light scattering ((DLS), Malvern Zetasizer Nano ZSP, Worcestershire, UK). Samples were prepared in a 1 mL quartz cuvette by diluting 50 μL of the sample with 950 μL of PBS. The samples were equilibrated in the instrument to 25° C. for 5 minutes prior to taking the measurement with the 633 nm laser. Zeta potential was measured in a similar method, but a Malvern Zetasizer dip cell adapter was used. The data were obtained with measurements performed at least in triplicate for all samples.

The particle size distribution (Z-average (d. nm)) and PDI were also measured in-line after formation as a part of the continuous manufacturing system using a NanoFlowSizer ((NFS), InProcess-LSP, Oss, Netherlands) with the inline sample module. The NFS utilizes a broadband light source with a center wavelength of 1300 nm. The data were obtained continuously during the manufacturing of each formulation and averaged over the duration of each run.

Three different liposome formulations were produced using continuous manufacturing. The first formulation (Lip-DTX) contained only DTX, the second formulation (Lip-DOX) contained only DOX, and the third formulation (Lip-DTX/DOX) contained both DTX and DOX. Each formulation was measured for its particle size distribution during manufacturing using the NFS and offline using Malvern DLS. The differences between the mean particle size and PDI measured using the NFS and DLS (Table 2) are a result of the measurement conditions and the instrumentation. The NFS utilizes a laser centered around 1300 nm, while the Malvern ZSP, which was used for the DLS measurements, contains a laser center around 633 nm. Since the measured particles were much smaller than the wavelengths of both lasers, Rayleigh scattering of the particles was the dominant factor used for calculation by the two instruments and lead to the differences in the measured particle size distributions. Furthermore, the samples were in motion and contained 5% ethanol when the NFS measurement was taken because it is an integrated process analytical technology within the continuous manufacturing platform. Conversely, the DLS measurements were conducted with static samples that were 50 times more dilute than the samples used for the NFS measurements. All particle size distribution measurements of the formulations, regardless of the measurement technique, showed that the liposomes were in a desirable size range (about 100 nm diameter) and had good homogeneity indicated by the low PDI (≤0.1) of the formulations. The Lip-DOX formulation had the most negative zeta potential, but all three formulations had a negative potential due to the phosphate diester moiety that is present in the formulations due to the addition of DSPE-PEG.

TABLE 2

| | NFS | | DLS | | |
|---|---|---|---|---|---|
| | Particle Size | | Particle Size | | Zeta |
| | (d · nm) | PDI | (d · nm) | PDI | Potential |
| Formulation | Mean ± SD | Mean ± SD | Mean ± SD | Mean ± SD | (mV) |
| Lip-DTX | 108.1 ± 3.2 | 0.075 ± 0.014 | 103.1 ± 1.5 | 0.060 ± 0.015 | −17.7 ± 3.5 |
| Lip-DOX | 88 ± 2.5 | 0.060 ± 0.011 | 85.9 ± 2.1 | 0.054 ± 0.012 | −24.2 ± 0.9 |
| Lip- | 101 ± 2.4 | 0.072 ± 0.015 | 97.6 ± 2.2 | 0.065 ± 0.008 | −20.2 ± 2.8 |
| DTX/DOX | | | | | |

For Table 2, Particle size (d. nm) and PDI were measured for each formulation in-line using the NFS and offline using the DLS. Zeta potential (mV) was measured using a dip cell adapter with the DLS instrument. Each sample was measured at least in triplicate.

### Example 3: Formulation Characterization—Cryo-TEM Preparation and Morphology Analysis

The morphological characteristics of the formulations were imaged using a cryogenic transmission electron microscope (cryo-TEM). C-flat 1.2/1.3 (300 mesh) and Ultrathin carbon 1.2/1.3 (300 mesh) grids (EMS) were subjected to glow discharge for 20 s (−15 mA) and 30 s (−20 mA), respectively, using an Easi-Glow cleaning system (Pelco). Samples were thawed on ice before use. 4 μl of sample was placed on a grid. The grid was back-blotted with filter paper for 3-4 seconds and frozen in the liquified ethane/propane mixture. All the grids were subsequently stored in a prelabelled grid box in liquid nitrogen. The images were recorded at a defocus of −2.5 μm on a K3 direct detector camera (Gatan) operating in counting mode with pixel size at 2.27 Å (17.5k) or 0.87 Å (45k). Images were collected with a total electron dose of about 40 e−/Å² for each image. Samples were shipped overnight and processed by the facility. Morphology and particle size (Z-average (d. nm)) were assessed from the images of the samples.

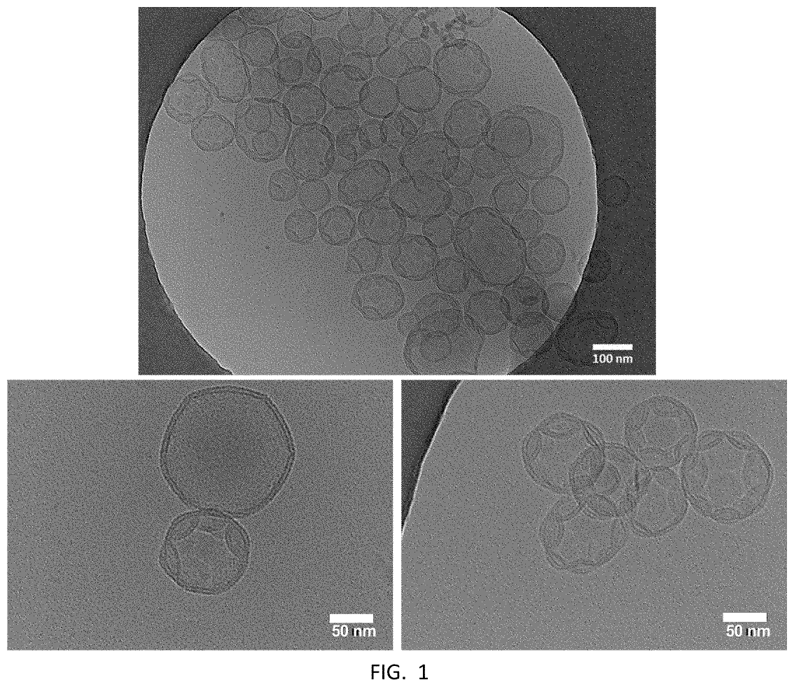

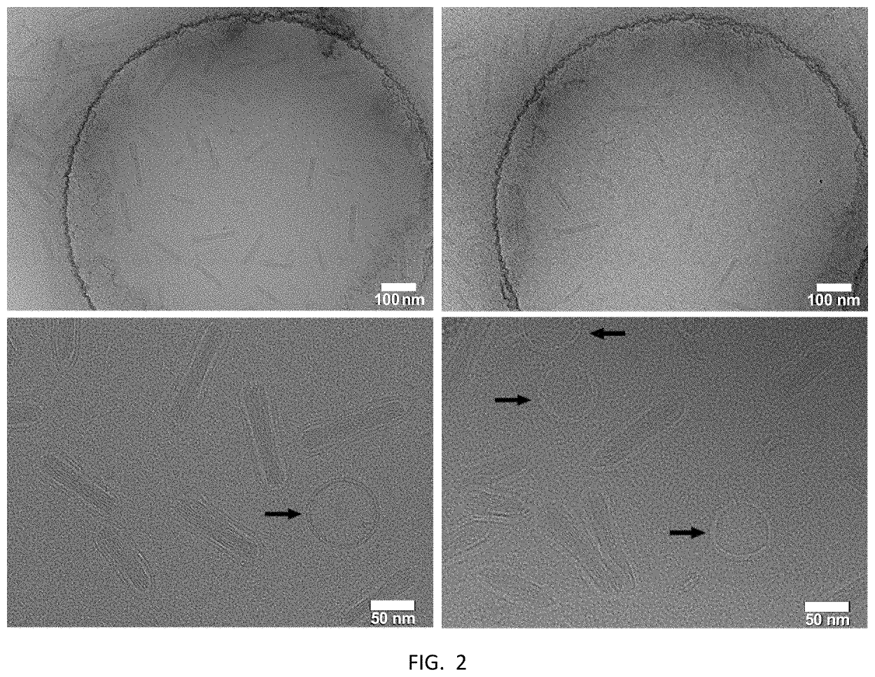

Cryo-TEM was used as a direct imaging technique to investigate the morphological characteristics of the formulations (FIG. 1). The particle size of the liposomes, as measured from the cryo-TEM images, was about 3-5 nm larger than the particle size measured using DLS. The fact that DLS is an intensity-based technique and cryo-TEM is a number-based technique can account for the differences in the particle size. The particles in the Lip-DTX formulation in FIG. 1 displayed a single intact bilayer with a dimple-like characteristic which can be indicative of the hydrophobic drug being embedded in that region. Additionally, the particles in the Lip-DTX/DOX formulation displayed an elongated structure (FIG. 2). This structure is due to the formation of DOX crystals in the aqueous compartment of the liposomes causing stretching into an elongated state. An average aspect ratio of about 1.5 was calculated for the elongated liposomes by dividing the major axis by the minor axis. Despite their elongated nature, the lipid bilayer of the particles did not appear to be compromised. Not to be limited by theory, the DTX may be acting as a stabilizer for the membrane and causing it to become more fluid which in turn would accommodate the longer DOX crystal formation within the aqueous core of the liposomes. The Lip-DOX formulation had relatively spherical shaped particles with defined bilayers and an obvious core of crystallized DOX as indicated by a dark band in the center of the liposomes.

### Example 4: Formulation Characterization—Lipid Components Assay

The lipid components were analyzed using a Waters Acquity Classic binary UPLC system (Waters Corporation, Wilmington, DE) comprised of a binary pump, online degasser, autosampler, and a thermostated column compartment. The column output from the UPLC was connected to the diverter valve on a Thermo Scientific Corona VEO RS Charged Aerosol Detector (CAD, Thermo Fisher Scientific, Waltham, MA). The mobile phase consisted of (A) Milli-Q water and (B) MeOH:ACN (75:25, v/v). An isocratic program was used at a ratio of 12% A and 88% B. The flow rate was 0.240 mL/min for a total run time of 10 minutes. An Acquity UPLC BEH C8 column (130 Å, 100 mm×1.0 mm, 1.7 μm) was used at a temperature of 45° C. as the stationary phase. The injection volume was 2 μL, and samples were held at 20° C. in the autosampler tray. The weak wash consisted of IPA:H2O (50:50, v/v), and the strong wash consisted of only IPA. Lipid components were detected using the CAD with a data collection rate of 20 Hz and a power function of 1. Filtered nitrogen was used for the system at a preset pressure of 61.6 psi. The nebulizer temperature was set to 45° C. Waters Empower 3 software (Waters Corporation, Wilmington, DE) was used for data acquisition and analysis.

Quantification of Lipid Components

Stock solutions of HSPC, cholesterol, and DSPE-PEG2000 were prepared in methanol and then diluted with methanol (w/w) to achieve the concentrations listed in Table 4. All liposomal samples were diluted 1:25 with methanol (w/w) to achieve concentrations in an appropriate range for quantification using CAD. All standards and samples were assessed in triplicate. The validation of the UPLC method was completed in accordance to the International Conference of Harmonization (ICH) Q2A and Q2B guidelines for analytical method development.

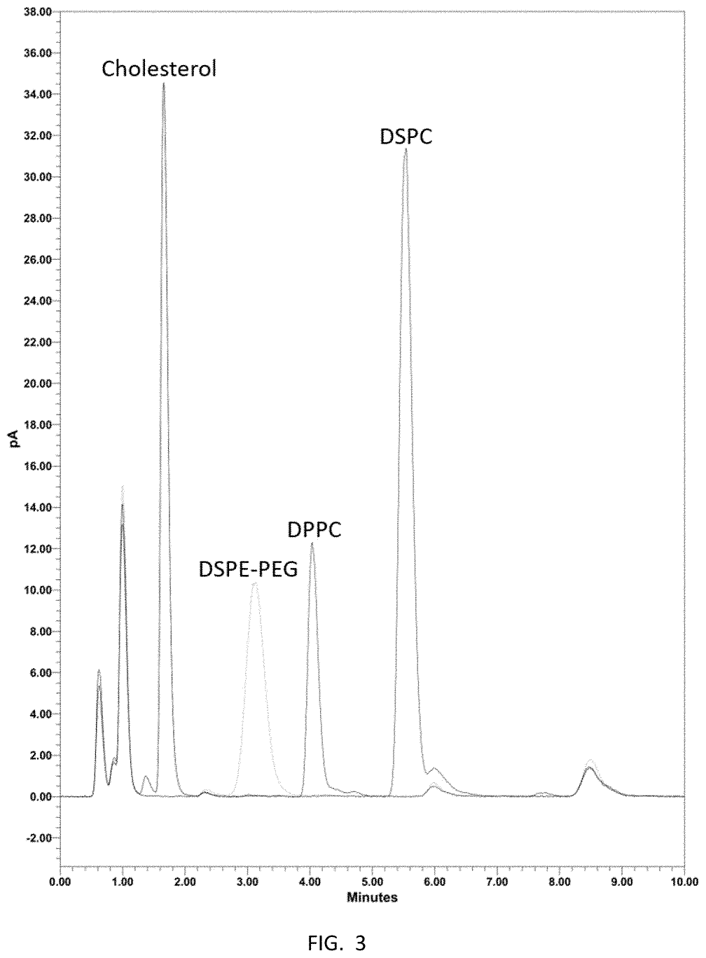

A novel UPLC-CAD method was developed to quantify the lipid components in the formulations. The linearity of each component was assessed in triplicate, and good linearity with high correlation coefficients (>0.99) was achieved for all lipid components (Table 4). The HSPC lipid is a mixture of two lipids, DPPC and DSPC, which were both separated with good correlation (>0.99). The precision of the method was assessed at two different levels: repeatability and intermediate precision. Repeatability was evaluated using three levels with three repetitions for each lipid component. The relative standard deviation (RSD) was <2% for the area and retention time for all the components and satisfied the ICH acceptance criteria for good analytical methods. Intermediate precision was assessed for each lipid component by testing three samples on different days. The intermediate precision of the system was <2% indicating the good precision of the UPLC-CAD method. The accuracy of the method was evaluated by testing three known concentrations with three replicates for each lipid component and quantifying the percent recovery. The assessed samples were different concentrations from those used to prepare the calibration curves. The recovery for all the samples was <5% indicating good accuracy for the analytical method. The good separation of the lipid components can be seen in FIG. 3 below. The three formulations were analyzed for their lipid components (Table 3). While it has been shown that there is no significant difference in pharmacokinetics of liposomes incorporating DPSE-PEG through the pre-insertion method or the post-insertion method, to prevent competition between DTX and DSPE-PEG during liposome formation and for easier processing with our continuous manufacturing platform, all formulations incorporated DSPE-PEG using the post insertion method. The concentration of DSPE-PEG was the least in the Lip-DTX formulation possibly due to DSPE-PEG and DTX competing for the same region within the liposomes. This was confirmed by the zeta potential measurements which showed that the formulations with DTX (Lip-DTX and Lip-DTX/DOX) had a less negative potential than the formulation without DTX (Lip-DOX). In addition, the final molar ratios of the lipid components remained relatively unchanged from the initial molar ratios for each formulation.

TABLE 3

| | Cholesterol Conc. | DSPE-PEG Conc. | HSPC Conc. |
|---|---|---|---|
| | (mg/mL) | (mg/mL) | (mg/mL) |
| | mean ± SD | mean ± SD | mean ± SD |
| Lip-DTX | 1.49 ± 0.23 | 0.195 ± 0.045 | 5.75 ± 0.19 |
| Lip-DOX | 1.51 ± 0.19 | 0.225 ± 0.029 | 5.82 ± 0.26 |
| Lip- | 1.50 ± 0.15 | 0.215 ± 0.036 | 5.83 ± 0.23 |
| DTX/DOX | | | |

For Table 3, the measured concentrations of lipid components for each formulation are shown. The standard deviation was obtained from triplicate samples.

### Example 5: Formulation Characterization—Quantification of Doxorubicin (DOX) and Docetaxel (DTX)

The drug encapsulation of DOX and DTX was quantified using a UPLC-UV method. The encapsulation efficiency (EE) was calculated according to the following equation and results listed in Table 4:

$$E E ( \% ) = \frac{c o n c e n t r ation of encapsulated API entrapped in liposomes}{c o n c e n t r ation of total API in the formulation} * 100 \%$$

TABLE 4

| Lipid | Concentration | Correlation | y-inter- | LOD | LOQ |
|---|---|---|---|---|---|
| Component | (μg/mL) | Coefficient | cept | (μg/mL) | (μg/mL) |
| HSPC | 125 | R² = 0.998 | 1.02 | 0.952 | 2.88 |
| | 62.5 | | | | |
| | 31.25 | | | | |
| | 15.625 | | | | |
| | 7.8125 | | | | |
| Cholesterol | 125 | R² = 0.999 | 9.56e−1 | 0.795 | 2.40 |
| | 62.5 | | | | |
| | 31.25 | | | | |
| | 15.625 | | | | |
| | 7.8125 | | | | |
| | 3.90625 | | | | |
| DSPE- | 50 | R² = 0.999 | 9.97e−1 | 0.481 | 1.45 |
| PEG2000 | 25 | | | | |
| | 12.5 | | | | |
| | 6.25 | | | | |
| | 3.125 | | | | |
| | 1.5625 | | | | |

For Table 4, the Limit of Detection (LOD) was 3.3*σ/S (where S is the slope and σ is the standard deviation of the y-intercept calculated from the calibration curve). The Limit of Quantification (LOQ) was 10*σ/S (where S is the slope and σ is the standard deviation of the y-intercept calculated from the calibration curve).

The total concentration of the API was determined by diluting 100 μL of sample into 2.5 mL of MeOH (v/w) and heating at 65° C. for 15 minutes and then quantified using a UPLC-UV assay. The concentration of the encapsulated API was determined by first removing the unencapsulated drug using ultracentrifugation. Next, 500 μL of the sample was transferred into an Amicon-Ultra centrifuge filter device (MilliporeSigma, Burlington, MA) with a molecular weight cut-off of 100 kD. The samples were centrifuged twice at 14,500 revolutions per minute (RPM) for 7 minutes using 300 μL of DI water to wash the filter between the cycles. Lastly, the filter was inverted and centrifuged at 3,900 RPM for 4 minutes to collect the retentate, which was diluted into 2.5 mL of MeOH (w/w). The samples were transferred into UPLC vials for analysis.

The total and encapsulated drug concentrations were quantified using the same Waters Acquity Classic binary UPLC system (Waters Corporation, Wilmington, DE) described previously but with the column output connected to a Waters Acquity UPLC PDA detector (Waters Corporation, Wilmington, DE). An Acquity UPLC BEH C18 column (130 Å, 100 mm×2.1 mm, 1.7 μm) held at 45° C. was used for the separation of both DOX and DTX. The mobile phase consisted of (A) Milli-Q water with 0.3% ortho-phosphoric acid 85% (v/v), and (B) ACN. Using a flow rate of 0.3 mL/min, the mobile phase composition started at 20% B and then was increased to 100% B in 3 minutes as the first step. At 4 minutes, the mobile phase composition was decreased back to the original composition of 20% B and held for 1 minute for a total run time of 5 minutes. The samples were held at 23° C., and the injection volume was fixed at 3 μL. The concentration of DOX and DTX in the samples were analyzed at 254 nm and 229 nm, respectively. The concentrations of the API in the samples were compared with reference standard curves prepared using serial dilution. Concentrations of 5 mg/mL of DTX in MeOH (w/w) and 3 mg/mL DOX in DI water (w/v) were prepared as stock solutions for the reference standard curves. Quality control standards were made from a fresh set of stock solutions to determine the accuracy of the standard curve. All standards and samples were assessed in triplicate.

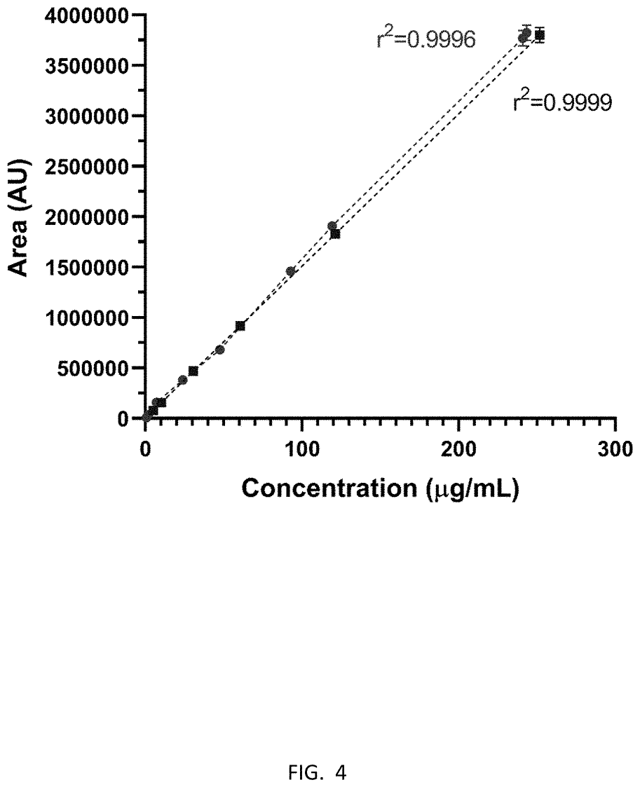

A novel UPLC-UV method was developed to measure the concentration of DTX and DOX in the liposomal formulations. The method was validated for its accuracy and precision using the same criteria described for the UPLC-CAD method. The calibration curves (FIG. 4) showed good linearity with high correlation coefficients (>0.99) thus confirming the UPLC-UV method suitable for quantification of the API. The encapsulation efficiency for each compound in the three formulations is displayed in Table 5. The encapsulation efficiency for DOX in the Lip-DOX formulation and the Lip-DTX/DOX formulation was higher than that of commercially available liposomal doxorubicin chemotherapy (e.g., Doxil). The multi-drug formulation achieved higher encapsulation efficiency for DTX and similar encapsulation efficiency for DOX as compared to the single drug formulations, indicating an effective manufacturing process to produce the co-encapsulated liposomal formulation.

TABLE 5

| | DTX | DOX |
|---|---|---|
| | encapsulation | encapsulation |
| | efficiency (%) | efficiency (%) |
| | mean ± SD | mean ± SD |
| Lip-DTX | 78.5 ± 3.4 | — |
| Lip-DOX | — | 98.2 ± 2.3 |
| Lip-DTX/DOX | 80.2 ± 2.8 | 97.1 ± 1.8 |

For Table 5, the encapsulation efficiency (%) of the DTX and DOX APIs are shown. Measurements were performed in triplicate.

### Example 6: In vitro Cytotoxicity Assay

Cervical cancer cells (HeLa cells) and chronic myelogenous leukemia cells (K562 cells) were purchased from Sigma Aldrich. The cells were cultured in T-75 bottles containing RPMI-1640 medium supplemented with 10% FBS and 1% antibiotics (Penicillin G-Streptomycin sulfate) at 37° C. in 5% CO₂ under humidified conditions. The isolated cell lines were seeded on 96-well plates (flat-bottomed; Corning) at a cell density of 10⁴ cells/well. After incubating for 24 hours, the cells were exposed to treatments containing free DTX, free DOX, free DTX+DOX mixture, liposomal DTX, liposomal DOX, and co-encapsulated liposomal DTX+DOX. The molar ratio of DTX to DOX was fixed at 1:3. The concentration of DOX ranged from 0.2 μg/mL to 1000 μg/mL, and the concentration of DTX was adjusted accordingly in the treatment assay. After 48 hours, 20 μL of MTT solution (5 mg/mL) was added to each well for a final concentration of 0.5 mg/mL and incubated for an additional 4 hours. Next, the culture media was slowly aspirated, and 100 μL of DMSO was added to each well to solubilize the formazan crystals. Viable cells were quantified by measuring the absorbance at 570 nm with a reference wavelength of 600 nm using an Infinite M200 microplate reader (Tecan Group Ltd., Männedorf, Switzerland).

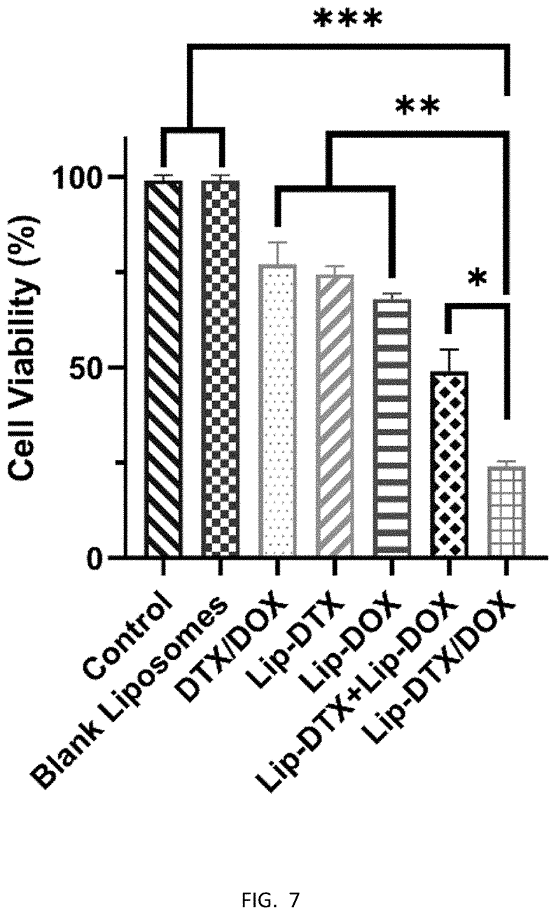

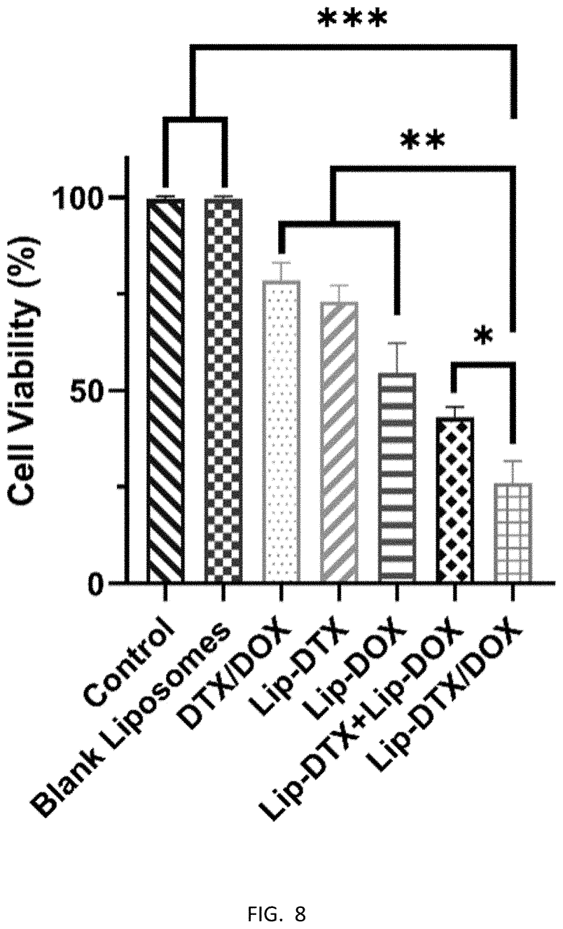

To evaluate the antitumor efficacy of the multidrug-loaded formulation, a standard cell viability assay was conducted on two different cell lines: HeLa (FIGS. 7) and K562 (FIG. 8). The results showed that the Lip-DTX/DOX formulation had a significant (p<0.001) impact on cell viability when compared to the control and blank liposomes in both cell lines. Additionally, the Lip-DTX/DOX formulation showed significantly (p<0.05) higher cytotoxicity than free DTX/DOX, Lip-DTX, or Lip-DOX. This indicates that more DTX and DOX were delivered by the liposomal formulations, and the internalization of multiple APIs was more effective in causing cell death in both the HeLa and K562 cell lines. Additionally, it is advantageous to combat drug resistance that the two API in the multi-drug formulation cause cell death via two different mechanisms.

### Example 7: Stability Study

Stability during transportation and storage is very important for a liposomal formulation. The storage stability of Lip-DTX, Lip-DOX, and Lip-DTX/DOX was performed at 4 ° C. using glass vials with crimped rubber stoppers. Particle size, PDI, and leakage stability of the liposomes were monitored for three months. At scheduled time intervals, 1 mL of sample was drawn from the storage containers and analyzed using DLS and UPLC-UV with the methods mentioned above. All measurements were conducted with at least n=3 for each time interval.

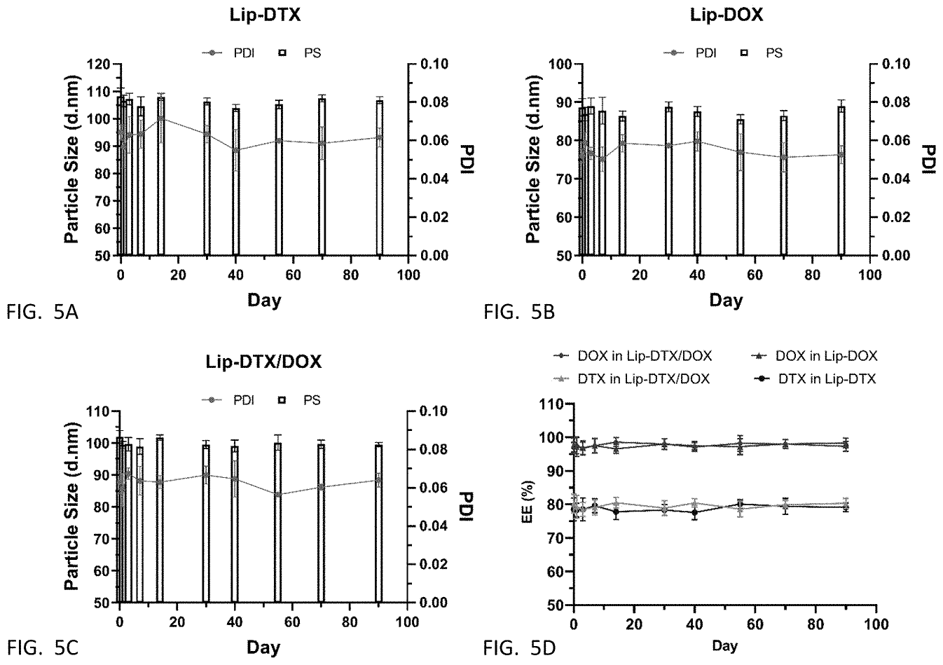

These drugs are typically administrated to patients via intravenous infusion; therefore, it was important to test their stability after dilution. Each formulation was diluted with saline, a typical diluent utilized in the clinic, at a ratio of 1:10 (v/v). All three formulations showed no statistical (p>0.05) difference in the particle size, PDI, or encapsulation efficiency as a result of dilution. All three formulations displayed physical and chemical stability for the duration of the study (90 days) stored in glass vials at 4° C. (FIG. 5A-5D). The encapsulation efficiency of both APIs did not significantly (p>0.05) change during the testing period.

Generally, a zeta potential value of <−30 mV or >30 mV has been shown to prevent aggregation between particles. However, the smaller observed zeta potential values (−17 to −24 mV; Table 2) for these three formulations appear to have provided sufficient electric double layer thickness to help improve long-term stability. There was no significant (p>0.05) change in the zeta potential throughout the duration of the long-term study, which helps to confirm chemical and physical stability of the formulations. The lipid component analysis showed relatively modest amounts of DSPE-PEG in the formulations (0.195 to 0.225 mg/mL; Table 3), most of the DSPE-PEG was present on the liposome surface as a result of manufacturing via the post-insertion method. Not to be bound by theory, but the combination of lipids used, the production of monodispersed particles or particles of low polydispersity, the continuous manufacturing method, and the presence of a majority of the PEG on the surface of the liposomes all likely contributed to the long-term stability of the formulations.

### Example 8: In Vitro Release Study

The release study was conducted using a dialysis method with PBS as the buffer media at pH 5.5, 6.5, and 7.4 to simulate the endosomal, tumoral, and plasma conditions, respectively. The pH was adjusted using 1 M HCl solution or 1 M NaOH solution as needed to achieve the desired media pH. Briefly, 5 mL of each formulation were placed in Spectrum Float-A-Lyzer G2 dialysis tubes (Thermo Fisher Scientific, Waltham, MA) with a molecular weight cut-off of 100 kD, which were then immersed in 50 mL of release media. The containers were incubated at 37° C. under shaking at 150 rpm for 96 hours. At 0-, 1-, 2-, 4-, 8-, 12-, 24-, 36-, 48-, 72-, and 96-hour intervals, 500 μL of the sample solution was withdrawn from the release media and replaced with an equal amount of fresh medium. The samples were analyzed using the UPLC-UV method previously described. The error bars were obtained by measuring each sample in triplicate.

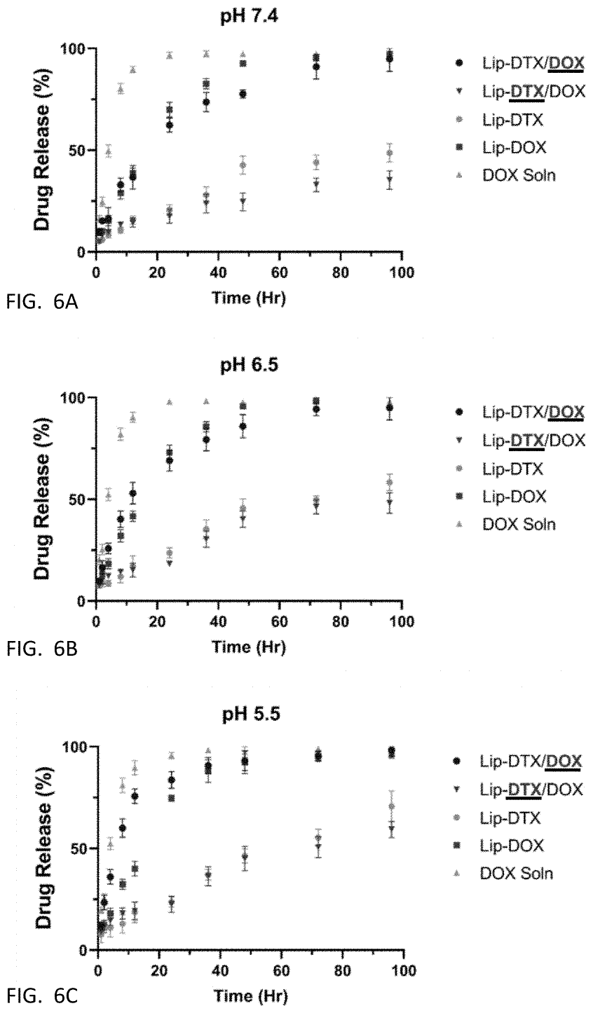

The three formulations were compared to DOX solution (1 mg/mL) at three different pH values. About 80% of the DOX passed through the Float-a-Lyzer membrane within 4 hours and nearly 100% by 24 hours. FIGS. 6A-6C show the in vitro drug release profiles for the formulations containing a single API at pH 5.5 (FIG. 6C), 6.5 (FIG. 6B), and 7.4 (FIG. 6A). The release of DTX was less than 10% for the first four hours for all three media. Although the release of DTX appeared slightly higher in the pH 5.5 medium at the 96-hr mark, there was no significant difference in drug release rate among the three media. The Lip-DTX achieved about a 70% release within the timeframe of the study. There was a burst release of DOX of about 20% for all samples within the first 4-hour period followed by a more sustained release profile. There appears to be a slight increase in the release rate of DOX as the pH of the media was decreased. However, there was no statical difference between the Lip-DOX release profiles in the three different pH media. The Lip-DOX formulation achieved nearly 100% drug release within the timeframe of the study.

The in vitro drug release profiles for Lip-DTX/DOX at the three different pH conditions is shown in FIG. 6A-6C. This formulation contains both APIs and accordingly two profiles are present for each media condition. In general, the release rate of DOX was slowest at pH 7.4 and the fastest at pH 5.5. In fact, at pH 5.5, the release rate of DOX was faster in Lip-DTX/DOX formulation compared to the Lip-DOX formulation. At pH 5.5 the carbonyl group of DTX binds to the hydrogen protons and causes the nitrogen atom on the molecule to undergo protonation. Not to be bound by theory, but this change in local charge density causes an increase in the steric hinderance between the DTX and the lipid components allowing for a faster release of the DOX from the internal compartment. The release of DTX from the Lip-DTX/DOX formulation in the pH 5.5 and 6.5 media appeared to have a biphasic release characteristic while the release profile in the pH 7.4 media did not. The higher pH appeared to provide insufficient surface charge and hydration of the particles to disrupt the Van der Waals interactions between the DTX and components of the lipid bilayer, such as cholesterol, resulting in a sustained release profile. The lower pH also can induce local membrane deformations which can also explain the increased rate of release of DOX from the co-encapsulated formulation.

There was minimal presence of unencapsulated APIs at time zero for all the formulation. This result confirms that liposomes were not significantly compromised during the manufacturing process or prior to conducting the release study. Additionally, this release data supports that the particles were also stable when the liposomes formed elongated structures to accommodate the DOX crystal growth process. The overall particle stability and pH dependent release of the Lip-DTX/DOX formulation suggests the formulation can retain the majority of the APIs during circulation and release most of the drugs at the microenvironment of the tumor where the pH is more acidic.

Materials

Doxorubicin (hydrochloride salt) (DOX) and docetaxel (DTX) were obtained from LC Laboratories (Woburn, MA). Hydrogenated soy L-α-phosphatidylcholine (HSPC) and 1,2-distearoylsn- glycero-3 phosphoethanolamine-N-[methoxy (polyethylene glycol)- 2000](DSPE-PEG) were obtained from Lipoid (Ludwigshafen, Germany). Cholesterol, phosphate-buffered saline (PBS, pH 7.4), ammonium sulfate, acetonitrile (ACN), isopropyl alcohol (IPA), and methanol with 10 mM ammonium formate (MeOH-AF) were purchased from Thermo Fisher Scientific Inc. (Waltham, MA). USP grade 190 proof ethanol (EtOH) was purchased from Sigma-Aldrich (St. Louis, MO). An in-house Milli-Q filtration system was used to obtain ultrapure water. Gibco RPMI-1640 growth media, Gibco fetal bovine serum (FBS), Gibco penicillin-streptomycin antibiotics (10,000 U/mL), Gibco trypsin-EDTA (0.25%), and 3-(4,5 dimethyl thiazolyl-2)-2,5-diphenyltetrazolium bromide (MTT) were purchased from Spectrum Chemicals Manufacturing Inc. (New Brunswick, NJ). All other chemicals used in this study were of analytical grade.

We claim:

1. A nanoparticle comprising:
(a) a first layer surrounding a core region;
(b) one or more particle modifiers present in or on the first layer; and
(c) one or more therapeutics present in the core region.

2. The nanoparticle of claim 1, wherein the nanoparticle comprises a polymer-based nanoparticle or lipid-based nanoparticle.

3. The nanoparticle of claim 1, wherein the first layer is an outer layer of the nanoparticle.

4. The nanoparticle of claim 1, wherein the particle modifier comprises one or more poorly water-soluble second therapeutic.

5. The nanoparticle of claim 4, wherein the poorly water-soluble second therapeutic comprises a taxane.

6. The nanoparticle of claim 5, wherein the taxane comprises docetaxel (DTX), or a pharmaceutically acceptable salt thereof.

7. The nanoparticle of claim 1, wherein the nanoparticle comprises a liposome.

8. The nanoparticle of claim 7, wherein the one or more particle modifiers is present in a phospholipid bilayer of the liposome.

9. The nanoparticle of claim 1, wherein the one or more therapeutic present in the core region comprises an amphipathic weak acid or base.

10. The nanoparticle of claim 9, wherein the one or more therapeutic present in the core region is selected from the group consisting of doxorubicin, daunorubicin, epirubicin, idarubicin, vincristine and irinotecan hydrochloride, or pharmaceutically acceptable salts thereof.

11. The nanoparticle of claim 1, wherein the one or more therapeutic present in the core region comprises a nucleic acid, an RNA, a mRNA, a microRNA, a shRNA, or an siRNA.

12. The nanoparticle of claim 1, wherein the nanoparticle comprises a liposome, wherein the liposome comprises
(a) a phospholipid bilayer surrounding a core region that is aqueous;
(b) one or more particle modifiers comprising docetaxel (DTX), or a salt thereof, present in a phospholipid bilayer of the liposome; and
(c) one or more therapeutics comprising doxorubicin (DOX), or a salt thereof, present in the aqueous core region of the liposome.

13. The nanoparticle of claim 1, wherein the one or more therapeutic, or salt thereof, present in the core region comprises crystalized one or more therapeutic or salt thereof.

14. The liposome of claim 7, wherein the liposome comprises phosphatidylcholine (PC), cholesterol, polyethylene glycol (PEG).

15. The liposome of claim 14, wherein a molar ratio of PC:cholesterol:PEG in the liposome is about 77:20:3.

16. A composition comprising a plurality of nanoparticles of claim 1 and one or more of a pharmaceutically acceptable carrier, a pharmaceutically acceptable excipient, or a pharmaceutically acceptable diluent.

17. A method for treating a cancer, comprising administering to a subject having cancer an amount effective to treat the cancer of the nanoparticle of claim 1, or a pharmaceutical composition thereof.

18. A method for making a nanoparticle, comprising:
(a) a first layer surrounding a core region;
(b) one or more particle modifiers present in or on the first layer; and
(c) one or more therapeutics present in the core region,
wherein the nanoparticle comprises a liposome, the method comprising:
dissolving lipid components and particle modifier, second therapeutic, or DTX or a salt thereof, in an organic solvent to produce a lipid-particle modifier-organic solvent solution;
injecting the lipid-particle modifier-organic solvent solution into an aqueous solution, forming a turbulent jet at the site where the lipid-organic solvent solution and the aqueous solution mix, forming liposomes;
concentrating the liposomes; and
mixing the concentrated liposomes with a solution of a first therapeutic or a salt thereof, at a temperature of about 70° C. or higher, or at about 70° C.

19. A liposome made according to the method of claim 18.

20. A nanoparticle comprising:
(a) a first layer surrounding a core region;
(b) one or more particle modifiers present in or on the first layer; and
(c) one or more therapeutics present in the core region,
wherein the nanoparticle is a liposome comprising one or more phospholipid bilayers, the one or more particle modifiers comprise taxanes and are present in or on a phospholipid bilayer of the liposome, the one or more therapeutics present in the core region comprise amphipathic weak acids or bases, and the liposome comprises one or more phosphatidylcholines (PC), one or more cholesterols, and one or more polyethylene glycols (PEG).
